(19) United States
(12) Patent Application Publication
(10) Pub. No.: US 2015/0190503 A1
(43) Pub. Date: Jul. 9, 2015

(54) METHODS OF USING CEO2 AND TIO2 NANOPARTICLES IN MODULATION OF THE IMMUNE SYSTEM

(71) Applicants: UNIVERSITY OF CENTRAL FLORIDA RESEARCH FOUNDATION, INC., Orlando, FL (US); SANOFI PASTEUR VAXDESIGN CORP., orlando, FL (US)

(72) Inventors: Brian Schanen, Orlando, FL (US); William Warren, Orlando, FL (US); William Self, Oviedo, FL (US); Sudipta Seal, Orlando, FL (US); Donald Drake, Orlando, FL (US)

(21) Appl. No.: 14/413,126

(22) PCT Filed: Jul. 8, 2013
(86) PCT No.: PCT/US2013/049578

§ 371 Date: Jan. 6, 2015

Related U.S. Application Data
(60) Provisional application No. 61/668,785, filed on Jul. 6, 2012.

(51) Int. Cl. A61K 39/39; C12N 7/00; A61K 39/245; A61K 9/14; A61K 33/24

(52) U.S. Cl. CPC A61K 39/39; A61K 9/14; A61K 33/24; A61K 39/245; C12N 7/00; A61K 2039/55505

(57) ABSTRACT

Redox-active NPs are disclosed that can potentiate innate immunity and stimulate distinct adaptive responses, producing distinct T cell subset polarization outcomes. Nanomaterials that can alter the cellular redox environment through ROS modulation can impact human immunology. TiO2 nanoparticles potentiate DC maturation, inducing the secretion of IL-12, p70, and IL-1B, while treatment with CeO2 nanoparticles induces IL-10, a hallmark of suppression. When delivered to T cells, the materials direct distinct T_{H} polarization, where TiO₂ stimulates largely a T_{H}1 dominated response, whereas CeO₂ stimulates a T_{H}2 bias and T_{Reg} differentiation.

### STATEMENT OF FEDERALLY SPONSORED RESEARCH AND DEVELOPMENT

This invention was made with government support under National Science Foundation grant number 0930170. The government has certain rights in this invention.

### TECHNICAL FIELD

The invention generally relates to methods of influencing immune systems and immune responses using redox-active nanoparticles.

### BACKGROUND OF INVENTION

Nanoparticles (NPs), particles sized between ˜1 and ˜200 nanometers, are now a ubiquitous aspect of modern life. For example, titanium dioxide (TiO₂) NPs are common additives to many consumer products, from food to paint to cosmetics (Weir et al. (2012) Environ. Sci. Technol., 46, 2242-50). However, questions remain as to how these materials affect human physiology. For example, some metallic NPs have been reported to induce acute toxicity in pulmonary and renal tissues (Fan & Alexeeff (2010) J. Nanosci. Nanotechnol. 10, 8646-57). Similar to foreign substances captured within the bloodstream or mucosa, NPs are likely to encounter a parallel fate, when, for example ingested or inhaled, where they will ultimately interact with the immune system. On the other hand, cerium oxide (CeO₂) NPs have shown promise in protecting tissues from oxidative stress and have been proposed for alleviating damage to surrounding healthy tissue following cancer radiation therapy (Patil et al. (2007) Biomaterials, 28, 4600-7; Colon et al. (2010) Nanomedicine 6, 698-705; Hirst et al. (2009) Small 5, 2848-56).

Testing of NPs can include many complexities. For example, testing NPs via oral exposure is multifaceted due to differences in diet, mucus secretion and composition, pH, gastrointestinal transit time, and even gastrointestinal flora comprising factors that can influence NP uptake (Fröhlich & Roblegg (2012) Toxicology, 291, 10-17). Active uptake mechanisms of NPs into cells have been studied. They include macropinocytosis, clathrin-mediated endocytosis and caveolae-mediated and non-clathrin, non-caveolae-mediated uptake. The latter can be subdivided into RhoA- (or IL-2Rβ-) dependent endocytosis, Cdc42/Arf1 or clathrin-independent cargo/glycophosphatidyl-inositol (GPI)-anchored protein enriched compartment-dependent (GEEC) endocytosis, Arf6-dependent endocytosis, and flotillin-dependent endocytosis (Fröhlich & Roblegg (2012) Toxicology, 291, 10-17).

There is growing interest in understanding how NPs might interact with cells of the immune system, given observations that immune cells act to eliminate or interact with NPs in the bloodstream (Dobrovolskaia et al. (2008) Mol. Pharm. 5, 487-95). Adding to this interest is the fact that nanoparticles have many different and tunable physicochemical properties, including size, shape, chemical composition, solubility, and surface chemistry that can influence their interaction with the immune system. There is the potential for NPs to have specific physiological effects on the cells of the immune system. The present invention is directed to aspects of the interactions between NPs and cells of the immune system, and other important goals.

### BRIEF SUMMARY OF INVENTION

The present invention is generally directed to redox-active nanoparticles (NPs) and their use in methods of modulating the immune system and immune responses. The redox-active NPs can be used in a variety of immunomodulatory applications, including inducing the production of distinct T cell subsets and polarization of T cell outcomes, and inducing the production cytokines by lymphocytes and dendritic cells. The NPs can also serve as effect adjuvants in vaccine formulations, to activate the induction of regulatory T cells and help induce tolerance, or to generally activate the immune system as could be of interest for in oncology.

The redox-active NPs of the present invention are cerium oxide (CeO₂) and titanium dioxide (TiO₂) nanoparticles. The disparate redox chemistries of these two types of NPs, i.e., the reductive activity of CeO₂ and the oxidative activity of TiO₂, impact the cellular redox environment and lead to immunomodulation. The aspects of the invention described herein each stem from the abilities of these nanoparticles to modulate specific elements of the immune system.

In a first aspect, the present invention is directed to methods of modulating an immune response in a subject comprising administering a pharmaceutical formulation comprising nanoparticles of CeO₂, or TiO₂, or both, to a subject in need thereof in an amount sufficient to modulate an immune response in the subject. In certain embodiments of this aspect, the production of antigen presenting cells is modulated, such as dendritic cells. In other certain embodiments, the production of CD4⁺ T helper cells is modulated or the production of CD8⁺ T helper cells is modulated. In further certain embodiments, nanoparticles of TiO₂ are administered and a T_{H}1-type immune response is modulated, wherein the modulation may be stimulation or suppression. In additional certain embodiments, nanoparticles of CeO₂ are administered and a T_{H}2-type immune response is modulated, wherein the modulation may be stimulation or suppression.

In a second aspect, the present invention is directed to methods of inducing a T_{H}2 T cell response in a subject comprising administering a pharmaceutical formulation comprising nanoparticles of CeO₂ to a subject in an amount sufficient to induce a T_{H}2 T cell response in the subject. In certain embodiments of this aspect, the T_{H}2 T cell response is a T_{H}2-polarized T cell response. In other certain embodiments, the T_{H}2 T cell response is production of T_{H}2 T cell cytokines.

In a third aspect, the present invention is directed to methods of inducing dendritic cell (DC) cytokine production in a subject comprising administering a pharmaceutical formulation comprising nanoparticles of CeO₂ to a subject in an amount sufficient to induce DC cytokine production in the subject. In certain embodiments of this aspect, DCs are induced to produce one or more of the following cytokines: IL-10, IL-1beta, IL-6, IL-7, IL-12 (p70), IL-15, IL-18, TNF-alpha, TGF-beta.

In a fourth aspect, the present invention is directed to methods of inducing T_{H}2 T cell cytokine production in vitro comprising adding nanoparticles of CeO₂ to an in vitro co-culture of DCs and T cells, thereby inducing T_{H}2 T cell cytokine production. In certain embodiments of this aspect, production of one or more of the following cytokines in induced: IL-4, IL-5, IL-9, IL-10, and IL-13.

In a fifth aspect, the present invention is directed to methods of inducing a T_{Reg} T cell response in a subject comprising administering a pharmaceutical formulation comprising nanoparticles of CeO₂ to a subject in an amount sufficient to induce a T_{Reg} T cell response in the subject.

In a sixth aspect, the present invention is directed to methods of inducing a T_{H}1 T cell response in a subject comprising administering a pharmaceutical formulation comprising nanoparticles of TiO₂ to a subject in an amount sufficient to induce a T_{H}1 T cell response in the subject. In certain embodiments of this aspect, the T_{H}1 T cell response is a T_{H}1-polarized T cell response. In other certain embodiments, the T_{H}1 T cell response is production of T_{H}1 T cell cytokines.

In a seventh aspect, the present invention is directed to methods of inducing T_{H}1 T cell cytokine production in a subject comprising administering a pharmaceutical formulation comprising nanoparticles of TiO₂ to a subject in an amount sufficient to induce T_{H}1 T cell cytokine production in the subject. In certain embodiments of this aspect, production of one or more of the following cytokines in induced: IFN-γ, IL-2, and TNF-β.

In an eighth aspect, the present invention is directed to methods of inducing dendritic cell (DC) cytokine production in a subject comprising administering a pharmaceutical formulation comprising nanoparticles of TiO₂ to a subject in an amount sufficient to induce DC cytokine production in the subject. In certain embodiments of this aspect, DCs are induced to produce one or more of the following cytokines: IL-1beta, IL-6, IL-7, IL-12 (p70), IL-15, IL-18, TNF-alpha.

In a ninth aspect, the present invention is directed to methods of treating an autoimmune disease in a subject comprising administering a pharmaceutical formulation comprising a therapeutically effective amount of nanoparticles of CeO₂ to a subject in need thereof thereby treating an autoimmune disease in the subject. In embodiments of this aspect, the autoimmune disease may be, but is not limited to, one or more diseases selected from the group consisting of diabetes, multiple sclerosis, and rheumatoid arthritis, autoimmune thyroiditis, Hashimoto's thyroiditis, Graves' ophthalmopathy, Lyme arthritis, reactive arthritis, contact dermatitis (nickel), psoriasis vulgaris, erythema nodosum, primary biliary cirrhosis, pulmonary sarcoidosis, and Crohn's disease.

In a tenth aspect, the present invention is directed to methods of modulating the immune system of a subject having cancer during a cancer treatment comprising administering a pharmaceutical formulation comprising a therapeutically effective amount of nanoparticles of CeO₂, or TiO₂, or both, to a subject undergoing treatment for cancer thereby modulating the immune system of a subject having cancer. In certain embodiments of this aspect, the modulation enhances the efficacy of the cancer treatment.

In an eleventh aspect, the present invention is directed to methods of treating allergic inflammation in a subject comprising administering a pharmaceutical formulation comprising a therapeutically effective amount of nanoparticles of CeO₂, or TiO₂, or both, to a subject in need thereof thereby treating allergic inflammation in the subject.

In a twelfth aspect, the present invention is directed to a vaccine formulation comprising (i) an antigen component and (ii) an adjuvant component, wherein the adjuvant component comprises nanoparticles of CeO₂, or TiO₂, or both. In a particular embodiment of this aspect, the adjuvant component comprises nanoparticles of CeO₂ and the vaccine formulation induces a protective humoral immune response. In another particular embodiment, the adjuvant component comprises nanoparticles of TiO₂ and the vaccine formulation induces a protective cellular immune response. In a further particular embodiment, the adjuvant component comprises nanoparticles of TiO₂ and the vaccine formulation is anti-cancer vaccine. In each embodiment of this aspect, the antigen and the nanoparticles may be conjugated together in the vaccine formulation or may be unconjugated in the vaccine formulation.

In each of the aspects and embodiments of the invention, the pharmaceutical formulation comprising nanoparticles of TiO₂ comprises between about 10 μg/ml and 100 μg/ml nanoparticles. Similarly, in each of the aspects and embodiments of the invention, the pharmaceutical formulation comprising nanoparticles of CeO₂ comprises between about 10 μg/ml and 100 μg/ml nanoparticles.

In each of the aspects and embodiments of the invention practiced in vitro, the amount of nanoparticles of TiO₂ added to an in vitro culture comprises between about 10 μg and 100 μg nanoparticles, and the amount of nanoparticles of CeO₂ added to an in vitro culture comprises between about 10 μg and 100 μg nanoparticles.

In each of the aspects and embodiments of the invention, the pharmaceutical formulation comprising nanoparticles of TiO₂ is a volume of between about 0.25 ml and 1.0 ml.

In each of the aspects and embodiments of the invention, the pharmaceutical formulation comprising nanoparticles may be administered to the subject via an intraperitoneal, intravenous, oral, or subcutaneous route.

In each of the relevant aspects and embodiments of the invention, one or more other active ingredients may be administered in conjunction with the pharmaceutical formulations comprising nanoparticles. The one or more other active ingredients may be included in the formulation comprising the nanoparticles or be present in a separate formulation. When two or more separate formulations are utilized, the formulations may be administered concurrently or consecutively, in any order. The one or more other active ingredients may be, but are not limited to, therapeutics, adjuvants and vaccines.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc., is provided for the purpose of illustration and description only and is by no means intended to define the limits the invention.

### BRIEF DESCRIPTION OF DRAWINGS

FIG. 1. CeO₂ NPs and TiO₂ NPs appear as soft agglomerates when diluted in X-VIVO 15 serum free media. High resolution transmission electron microscopy of (A) CeO₂ NPs indicates a composition of individual 3-5 nm nanocrystallites and (B) 7-10 nm TiO₂ (anatase) NPs. The average size distribution of (C) CeO₂ and (D) TiO₂ NPs were measured using dynamic light scattering following a 24 hour incubation of the prepared NP solutions (each at 500 mM) in X-VIVO 15. Selected area electron diffraction patterns (SAEDP) of the CeO₂ (E) and TiO₂ NPs (F) were carried out using a high-resolution transmission electron microscope (HRTEM) equipped with a FEI Tecnai F30 having an energy-dispersive X-ray (EDX) analyzer. The SAED pattern of CeO₂ NPs, where A(111), B(200), C(220) and D(311) correspond to the different lattice planes of CeO₂ and confirms the crystalline structure of this material. Similarly, the SAED pattern of TiO₂ also confirms the crystalline nature of the material since the A(101), B(004), C(200) and D(211) rings correspond to the different lattice planes of the NPs. Surface oxidation state of CeO₂ and TiO₂ NPs were calculated from the XPS spectrum of Ce3d (G) and Ti 2p (H). (G) Deconvoluted peaks at 882.36 eV, 898.20 eV, 901.23 eV, 907.03 eV, and 916.64 eV are attributed to a Ce4+ oxidation state (light gray solid line) while 880.22 eV, 885.24 eV, 899.16 eV and 903.68 eV are the characteristic peaks of a Ce3+ oxidation state (dark gray solid line). Intensity of the peaks for Ce3+ and Ce4+ were estimated, and Ce3+/Ce4+ ratio on the surface of the nanoparticles were calculated and found to be 1.66. (H) In the case of TiO₂ NPs, the binding energies of Ti 2p3/2 and Ti 2p1/2 are at approximately 458.84 eV and 464.62 eV, respectively. The difference of ˜5.8 eV in both peaks indicates a valence state of +4 for Ti on the surface of the NPs.

FIG. 2. CeO₂ NPs trigger human DCs to produce significant amounts of IL-10. (A) Dendritic cells were exposed to the indicated concentrations of NPs for 24 hrs and assessed for viability using 7-AAD and apoptosis by Po-Pro staining. As negative and positive controls, DCs were left untouched (mock) or treated with 1 mg/ml Fas ligand (FAS), respectively. Bar graph data are plotted as mean±SD. (B) Dendritic cells were exposed to the indicated concentrations of NPs for 24 hours and assessed for phenotypic expression of human DC markers, as indicated, by flow cytometric analysis. (C) Supernatants from DCs stimulated with 1 mM of either NPs were examined for soluble cytokines by Bio-Plex assay. Each dot on the scatter plot represents the signal for an individual donor; Data are mean+/−SD, n=10. A paired t-test was performed: **p<0.005, ***p<0.0005 versus TiO₂ or CeO₂ group; ^{oo} p<0.005, ^{ooo}p<0.0005 versus mock group.

FIG. 3. Human DCs have the capacity to internalize CeO₂ and TiO₂ NPs. Cytokine-derived human DCs were pulsed for 24 hours with the listed dosing range of either NP. The DCs were harvested and washed several times before examination by inductively coupled plasma-mass spectroscopy (ICP-MS) for metal analysis and detection (ppb). Each sample was examined for the presence of both cerium (bottom) and titanium (top) as an assay detection control. Ten donors were analyzed in total. The paired t-test was used for statistical analyses. n=10; **p<0.005, ***p<0.0005 versus mock group.

FIG. 4. Redox activities of NPs modulate ROS production in DCs and activate the NLRP3 inflammasome. (A) Human DCs were cultured in the absence or presence of the indicated treatment (for 24 h prior to being examined for ROS). (B) DCs were cultured in the presence of CeO₂ NPs at various concentrations for 8 h prior to the addition of H₂O₂ for the rest of the 24-h incubation. Oxidative stress was measured by DCF-DA fluorescence. Six donors were analyzed in total. (C) DCs were stimulated for 24 h with LPS (10 ng/mL) or alhydrogel (AlHy, 150 μg/mL) as a positive control for NLRP3 activation. Alternatively (D), TiO₂ or CeO₂ NPs were delivered at 1 μM to the cultures for 24 h prior to measurements for the presence of IL-1β in the presence or absence of the NLRP3 inhibitor glibenclamide (50 μM). Each data point is representative of an individual cell donor (n=10).

FIG. 5. T cell stimulatory properties of TiO₂ NPs and response suppression and induction of T_{Regs} by CeO₂ NPs. (A) CD4⁺ T cells were isolated and cultured in the absence or presence of 10 μM TiO₂ NPs, 10 μM CeO₂ NPs, PHA, 10 μM TiO₂ NPs with PHA, or 10 μM CeO₂ NPs with PHA, as indicated, for 5 days. (B) Naïve T helper cells were cultured in the presence of T cells untouched (mock), co-cultured with matured DCs (positive), or pulsed with CeO₂ NPs or TiO₂ NPs. The cultures were harvested on day 7 and stained for Foxp3⁺ expression (n=8 donors). (C) Reduced Fas expression in CeO₂ NPs+PHA-treated cultures, compared with PHA alone. T cell cultures were stained for surface expression of CD95 and assessed using flow cytometry. Data are plotted as histogram overlays for each condition. Plots are representative of three donors, each with similar response profiles.

FIG. 6. CeO₂ and TiO₂ NP-primed DCs differentially modulate CD4+ T cells proliferation. Naive CD4+ T cells were isolated and labeled with the division-sensitive dye, CFSE. (A) The CFSE-labeled T cells were then co-cultured for 5 days with immature DCs (iDCs; untreated), matured DCs (mDCs; treated overnight with TNFa and PGE2), or NP treated DCs (24 hour treatment with the indicated nanomaterial described on the x-axis). (B) Thereafter, the cells were harvested and examined for proliferating (CFSE-low; left panel) and activated (CD4+CD25+; right panel) T cells by flow cytometry, n=10.

FIG. 7. CeO₂ and TiO₂ NPs directly affect cytokine secretion by human CD4⁺ T cells following a 24-h incubation. Supernatants from the T cell stimulatory assays were examined for T_{H}1- and T_{H}2-associated cytokines using a Bioplex array. Each dot on the scatter plot represents the signal for an individual cell donor (n=10).

FIG. 8. IgG1 and IgG2c antibody responses to test formulations are shown.

FIG. 9. IL-5 cytokine responses to test formulations are shown.

FIG. 10. INFγ cytokine responses to test formulations are shown.

FIG. 11. IgG1 and IgG2a antibody responses to test formulations are shown.

FIG. 12. INFγ (A) and IL-5 (B) cytokine responses to test formulations are shown.

### DETAILED DESCRIPTION OF THE INVENTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

Upon exposure to an antigenic stimulus, the immune system reacts, in part, through differentiation of naïve T_{H} cells into two broad subsets of T helper cells, T_{H}1 and T_{H}2 cells, characterized by differences in cytokine production. T helper-1 (T_{H}1) cells produce IFN-γ, IL-2, and TNF-β and they are typically involved in cell-mediated immune responses that are beneficial in host defenses against intracellular pathogens and malignant cells, but detrimental in mediating autoimmunity. T_{H}1 cells also activate macrophages and elicit delayed-type hypersensitivity reactions. T_{H}1 responses are generally associated with anti-viral and anti-cancer immune reactions. T_{H}1 cells are also important for defending against infections with intracellular microbes.

T_{H}2 cells secrete IL-4, IL-5, IL-9, IL-10, and IL-13, which augment antibody responses, including IgE production, and protect against helminth infestations, but also cause allergy and asthma. IL-4, IL-5, and IL-10 are important for IgE production and suppress cell-mediated immunity. T_{H}2 responses are effective for clearing parasitic infections, such as the trematode parasite Schistosoma mansoni that causes schistosomiasis in humans, and are identified by high secretion of IL-4 and IL-13. A T_{H}2 cell predominance has been found in the skin of patients with chronic graft-versus host disease, progressive systemic sclerosis, systemic lupus erythematosus, and allergic diseases.

T_{H}1 and T_{H}2 responses are mutually antagonistic. They normally exist in equilibrium and cross-regulate each other to some degree. T_{H}1 and T_{H}2 cytokines oppose each other's function and typically exist in a balanced state. The cytokines produced by each cell subset act as their own autocrine growth factors but cross-regulate the other subset's development and function. This model of cross-regulation has been used to explain a number of in vivo immune phenomena.

An altered balance between T_{H}1 and T_{H}2 cytokines is thought to underlie the etiopathogenesis of many immune-mediated diseases. Indeed, the balance between T_{H}1 and T_{H}2 subsets determines susceptibility to malignant, infectious, allergic, and autoimmune diseases. For example, allergic inflammation is typically characterized by a T_{H}2 cell cytokine-like response, with overexpression of T_{H}2 cytokines, such as IL-4, IL-5, IL-13, and IL-25, and under-expression of T_{H}1 cytokines, such as IFN-γ. Also, evidence from animal models suggests that T_{H}1-type lymphokines are involved in the genesis of organ-specific autoimmune diseases, such as experimental autoimmune uveitis, experimental allergic encephalomyelitis, and insulin-dependent diabetes mellitus. Administration of T_{H}1 (e.g., IL-12) or T_{H}2 (e.g., IL-4) cytokines in vivo, thus can promote or inhibit autoimmunity. However, direct administration of cytokines to treat an autoimmune disease may not be feasible in the clinical setting because of the short half-life of the cytokines in vivo.

The T_{H}1/T_{H}2 paradigm, with its differentiated T_{H} subsets balancing each other, offers the possibility of interfering with diseases, such as autoimmune diseases, by affecting the balance. Several studies have reported that an imbalance in T_{H}1/T_{H}2 responses may impact the pathogenesis of pathological conditions, including autoimmune diseases (e.g., Druet et al. (1995) J. Exp. Immunol. 101 (suppl. 1), 9-12). Multiple control pathways of T_{H}1 and T_{H}2 cell development are now known, and understanding the dynamics and complexity of the in vivo regulation processes is advancing. Further, the notion that T_{H}1 and T_{H}2 responses are themselves controlled by another type of T cell, regulatory T cells or T_{Regs}, offers other opportunities for immunotherapeutic intervention in autoimmune and allergic conditions.

Various strategies have been proposed to modulate the T_{H}1/T_{H}2 balance (e.g., Dumont (2002) Expert Opin. Ther. Patents 12, 341-367). They include procedures that affect the differentiation of T_{H}1 and T_{H}2 cells, the production of effector cytokines, and the activities of the cytokines.

Specific means for modulating the T_{H}1/T_{H}2 balance are provided herein. Such means are based on the use of redox-active NPs to modulate immune responses and elements thereof by inducing production of distinct T cell subsets, such as T_{H}1 and T_{H}2 cells, and cytokines produced by such cells. Redox-active NPs are those NPs that possess reduction/oxidation (redox) activity. Most metal oxide particles have no redox potential. Because of the high difference in redox potential between oxidation states, most metal oxide particles are not redox-active at normal temperatures and pressures. However, for example, cerium oxide (CeO₂) nanoparticles (nanoceria) have redox activity because redox cycles between the Ce³⁺ and Ce⁴⁺ oxidation states allow them to react catalytically with, for example, superoxide and hydrogen peroxide (Celardo et al. (2011) Nanoscale 3, 1411-20). Because of the coexistence of Ce³⁺ and Ce⁴⁺ on the surface of CeO₂ nanoparticles, they are redox-active; as a result of the low redox potential between Ce³⁺ and Ce⁴⁺ (1.7 eV), they can switch back and forth. As shown herein, cerium oxide nanoparticles have catalytic activity and antioxidant properties in tissue culture and animal models. As further shown herein, titanium dioxide (TiO₂) nanoparticles also exhibit excellent properties.

As stated above, and described in detail below, the present invention is directed to redox-active NPs that can modulate immune responses, and to methods of using such NPs in the production of distinct T cell subset polarization. CeO₂ NPs were found to induce DCs to produce IL-10 and, when co-cultured with T cells, induced T_{H}2 cytokine production. Such responses are beneficial in adjuvants for vaccines targeting humoral immunity. TiO₂ NPs were found to induce DCs to produce IL-12 and stimulate a T_{H}1-polarized T cell response. Such responses are beneficial in adjuvants for vaccines relying on cellular immunity, such as cancer vaccines. CeO₂ NPs were also found to stimulate DCs to produce IL-10, making CeO₂ NPs useful in promoting T_{Reg} responses. Such responses are beneficial in treating autoimmune diseases and cancer. These redox-active NPs can modulate immune responses, producing distinct T cell subset polarization outcomes, likely due to their contrasting activity (TiO₂ NPs=oxidant; CeO₂ NPs=antioxidant).

The data provided herein demonstrate that surface reactivity can profoundly influence immune responses and directionality. Specifically, the data suggest that low doses of redox-active NPs can modulate the activation status of human DCs and alter the direction of CD4⁺ T helper cells in response to challenge. The data illustrate the capacity for NPs to influence T_{H} polarization in a distinct manner, influenced by parameters such as size, and therefore exposed surface area. NPs will be useful in driving a T cell-biased response in the direction necessary for prophylaxis and will also be useful, when conjugated to an antigen of interest, to stimulate and/or sustain a desired immune response to the antigen.

CeO₂ NPs will be useful for therapeutic vaccines and autoimmune and inflammatory disease prophylaxis, considering the importance of T_{H}2 responses for driving antibody production, a defining feature of prophylactic vaccination, coupled with the observation that CeO₂ NPs can aggregate (perhaps via intracellular DC transit) in lymph nodes (Cassee et al. (2011) Crit. Rev. Toxicol., 41, 213-29). In this context, modulation of these redox reactions by sustained CeO₂ NP treatment will also provide a therapeutic benefit, in the induction of T_{Regs}, in controlling inflammatory-mediated diseases.

### Characteristics of the Nanoparticles

The nanoparticles of the present invention are nanoparticles of TiO₂ and CeO₂. The NPs can be obtained from sources such as titanium isopropoxide or cerium nitrate hexahydrate, for example. Alternatively, the NPs can be produce according to the procedures provided in the Examples below.

When used in the methods and formulations of the present invention, at least about 50%, 55%, 60%, 65%, 70%, 75%, 80%, 85%, 90%, 95%, 96%, 97%, 98%, 99% or 100% of the CeO₂ NPs have an average diameter of between about 1 nm and 200 nm. The average diameter may also be between about 1 nm and 50 nm, between about 1 nm and 20 nm, between about 1 nm and 15 nm, between about 1 nm and 10 nm, between about 2 nm and 6 nm, or between about 3 nm and 5 nm, but is not limited to these specific ranges. In a particular embodiment, at least about 90% of the CeO₂ NPs have an average diameter of between about 3 nm and 5 nm.

Similarly, when used in the methods and formulations of the present invention, at least about 50%, 55%, 60%, 65%, 70%, 75%, 80%, 85%, 90%, 95%, 96%, 97%, 98%, 99% or 100% of the TiO₂ NPs have an average diameter of between about 1 nm and 200 nm. The average diameter may also be between about 1 nm and 50 nm, between about 3 nm and 20 nm, between about 5 nm and 15 nm, between about 6 nm and 12 nm, or between about 7 nm and 10 nm, but is not limited to these specific ranges. In a particular embodiment, at least about 90% of the TiO₂ NPs have an average diameter of between about 7 nm and 10 nm.

### Formulations Comprising Nanoparticles

When administered to a subject, the nanoparticles may be provided in a pharmaceutical formulation comprising the nanoparticles and a carrier. Examples of suitable carriers are well known to those skilled in the art and include water, water-for-injection, saline, buffered saline, dextrose, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes. The terms specifically exclude cell culture medium. The formulations may further comprise stabilizing agents, buffers, antioxidants and preservatives, tonicity agents, bulking agents, emulsifiers, suspending or viscosity agents, inert diluents, fillers, and combinations thereof.

The identity of the carrier(s) will also depend on the means used to administer pharmaceutical formulations comprising NPs to a subject. For example, pharmaceutical formulations for intramuscular preparations can be prepared where the carrier is water-for-injection, 0.9% saline, or 5% glucose solution. Similar carriers may be used for intravenous preparations. Pharmaceutical formulations may also be prepared as liquid or powdered atomized dispersions for delivery by inhalation. Such dispersion typically contain carriers common for atomized or aerosolized dispersions, such as buffered saline and/or other compounds well known to those of skill in the art. The delivery of the pharmaceutical formulations via inhalation has the effect of rapidly dispersing the NPs to a large area of mucosal tissues as well as quick absorption by the blood for circulation. One example of a method of preparing an atomized dispersion is described in U.S. Pat. No. 6,187,344, entitled, “Powdered Pharmaceutical Formulations Having Improved Dispersibility,” which is hereby incorporated by reference in its entirety.

Additionally, the pharmaceutical formulations may also be administered in a liquid form. The liquid can be for oral dosage, for ophthalmic or nasal dosage as drops, or for use as an enema or douche. When the pharmaceutical formulation is formulated as a liquid, the liquid can be either a solution or a suspension of the NPs. There is a variety of suitable formulations for the solution or suspension of the NPs that are well known to those of skill in the art, depending on the intended use thereof. Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents.

The pharmaceutical formulations may also comprise encapsulated NPs. Encapsulation facilitates access to the site of action and allows the administration of other active ingredients simultaneously.

### Means for Administration

The NPs and pharmaceutical formulations comprising NPs may be administered to a subject via means that include, but are not limited to, oral, sublingual, intranasal, intraocular, rectal, transdermal, mucosal, pulmonary, topical and parenteral administration. Parenteral modes of administration include without limitation, intradermal, subcutaneous (s.c., s.q., sub-Q, Hypo), intramuscular (i.m.), intravenous (i.v.), intraperitoneal (i.p.), and intra-arterial. Any known device useful for parenteral injection or infusion of drug formulations can be used to effect such administration. Means for administering nanoparticles to a subject via intraperitoneal, intravenous, oral, and subcutaneous routes are well known in the art (see., e.g., Hirst et al. (2011) Environ. Toxicol. [Epub ahead of print]; Umbreit et al. (2012) J. Appl. Toxicol. 32, 350-7; Patri et al. (2009) J. Appl. Toxicol. 29, 662-72).

### Amounts for Administration

The nanoparticles of the present invention are administered to a subject in an amount sufficient to induce a particular effect or in a therapeutically effective amount. Thus, individual or unit doses of the nanoparticles are administered to a subject to achieve the disclosed goals.

The individual or unit doses administered to a subject may be in a pharmaceutical formulation comprising the nanoparticles. The concentration of nanoparticles in an individual or unit dose administered to a subject when practicing the methods of the invention lies preferably within a range of circulating concentrations that include the ED₅₀ with little or no toxicity. The concentration may vary within this range depending upon the dosage form employed and the route of administration used. Generally, a concentration sufficient to induce a particular effect or a therapeutically effective amount will vary with the subject's age, condition, and gender, as well as the severity of the medical condition of the subject. The dosage may be determined by a physician and adjusted, as necessary, to suit observed effects of the treatment.

Generally, the concentration of nanoparticles in an individual or unit dose between about 1 μg/ml and 1000 μg/ml. Suitable ranges also include, but are not limited to, between about 1 μg/ml and 100 μg/ml, between about 10 μg/ml and 1000 μg/ml, between about 10 μg/ml and 100 μg/ml, between about 10 μg/ml and 50 μg/ml, between about 50 μg/ml and 100 μg/ml, and between about 25 μg/ml and 75 μg/ml. Particular concentrations in an individual or unit dose include about 1, 5, 10, 15, 20, 25, 30, 35, 40, 45, 50, 55, 60, 65, 70, 75, 80, 85, 90, 95, and 100 μg/ml of the nanoparticles.

The volume of a pharmaceutical formulation comprising the nanoparticles that may be administered to a subject includes, but is not limited to, between about 0.1 and 100 ml, between about 0.1 and 50 ml, between about 0.1 and 25 ml, between about 0.1 and 10 ml, between about 0.25 and 5 ml, between about 0.25 and 2.5 ml, and between about 0.25 and 1 ml. Particular volumes include, but are not limited to, about 0.1, 0.2, 0.3, 0.4, 0.5, 0.6, 0.7, 0.8, 0.9, 0.1, 1.25, 1.5, 1.75, 2.0, 2.25, 2.5, 2.75, or 3.0 ml, or more. The specific volume may vary based on the concentration of nanoparticles in the formulation.

Administration frequencies of the individual or unit doses will vary based on factors such as the purpose for the administration, the method being practiced, the volume to be administered and the concentration of the formulation, among other factors. Acceptable frequencies include 4×, 3×, 2× or once daily, every other day, every third day, every fourth day, every fifth day, every sixth day, once weekly, every eight days, every nine days, every ten days, bi-weekly, monthly and bi-monthly. Depending on the means of administration, the pharmaceutical formulations may be administered all at once, such as with an oral formulation in a capsule or liquid, or slowly over a period of time, such as with an intramuscular or intravenous administration.

The duration over which the methods of the present invention will continue to be practiced will be best determined by the attending physician. However, the methods may continue to be practiced for days, week, months or even years.

A therapeutically effective amount and an amount sufficient to achieve a particular stated goal can be measured by the therapeutic effectiveness of the compound. The dosages, however, may be varied depending upon the requirements of the patient, the severity of the condition being treated, and the NPs being used. In one embodiment, the therapeutically effective amount and an amount sufficient to achieve a stated goal is sufficient to establish a pre-selected maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50, 219-244 (1966) and Table 1 for equivalent surface area dosage factors).

TABLE 1
Equivalent Surface Area Dosage Factors

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

### Methods of Inducing a Response

In methods of the invention directed to inducing a particular response (e.g., a T cell response, T_{H}1 T cell response, T_{H}2 T cell response, DC cytokine production, T_{H}1 T cell cytokine production, T_{H}2 T cell cytokine production, T_{Reg} T cell response), the particular response is induced by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99% or even 100% in comparison to comparable circumstance in which NPs are not utilized.

### Methods of Treatment

The unique properties of the nanoparticles of CeO₂ and TiO₂ allow them to be used in the treatment of diseases and conditions mediated, at least in part, by components of the immune system. For example, administration of nanoparticles of CeO₂ can be used to treat an autoimmune disease in a subject, and the present invention includes methods of treating an autoimmune disease in a subject comprising administering a pharmaceutical formulation comprising a therapeutically effective amount of nanoparticles of CeO₂ to a subject in need thereof. The autoimmune disease that may be treated using these methods are not limited, and include one or more of diabetes, multiple sclerosis, and rheumatoid arthritis, autoimmune thyroiditis, Hashimoto's thyroiditis, Graves' ophthalmopathy, Lyme arthritis, reactive arthritis, contact dermatitis (nickel), psoriasis vulgaris, erythema nodosum, primary biliary cirrhosis, pulmonary sarcoidosis, and Crohn's disease.

Similarly, administration of nanoparticles of CeO₂ and TiO₂ can be used to modulate the immune system of a subject having cancer during a cancer treatment, particularly cancers associated with disregulation of the immune system. The present invention thus includes methods of modulating the immune system of a subject having cancer during a cancer treatment comprising administering a pharmaceutical formulation comprising a therapeutically effective amount of nanoparticles of CeO₂, or TiO₂, or both, to a subject undergoing treatment for cancer thereby modulating the immune system of a subject having cancer.

Administration of nanoparticles of CeO₂ and TiO₂ can further be used to treat allergic inflammation in a subject. The present invention thus includes methods of treating allergic inflammation in a subject comprising administering a pharmaceutical formulation comprising a therapeutically effective amount of nanoparticles of CeO₂, or TiO₂, or both, to a subject in need thereof.

As used herein, the terms “treat”, “treating”, and “treatment” have their ordinary and customary meanings, and include one or more of, ameliorating a symptom of a disease or condition in a subject, blocking or ameliorating a recurrence of a symptom of a disease or condition in a subject, decreasing in severity and/or frequency a symptom of a disease or condition in a subject. Treatment means ameliorating, blocking, reducing, decreasing or inhibiting by about 1% to about 100% versus a subject to which a pharmaceutical formulation comprising NPs has not been administered. Preferably, the ameliorating, blocking, reducing, decreasing or inhibiting is about 100%, 99%, 98%, 97%, 96%, 95%, 90%, 80%, 70%, 60%, 50%, 40%, 30%, 20%, 10%, 5% or 1% versus a subject to which a pharmaceutical formulation comprising NPs has not been administered.

### Vaccine Formulations

The vaccine formulations of the present invention comprise (i) an antigen component and (ii) an adjuvant component. In each of the vaccine formulations of the invention, the adjuvant component comprises nanoparticles of CeO₂, or TiO₂, or both.

Depending on the particular NP used as the adjuvant component, the vaccine formulation may be used to induce a protective humoral immune response, or a protective cellular immune response. Further, depending on the identity of the antigen component and the particular NP used as the adjuvant component, the vaccine formulation may be used as an anti-cancer vaccine.

Generally, the concentration of nanoparticles in a vaccine formulation is between about 1 μg/ml and 1000 μg/ml. Suitable ranges also include, but are not limited to, between about 1 μg/ml and 100 μg/ml, between about 10 μg/ml and 1000 μg/ml, between about 10 μg/ml and 100 μg/ml, between about 10 μg/ml and 50 μg/ml, between about 50 μg/ml and 100 μg/ml, and between about 25 μg/ml and 75 μg/ml. Particular suitable concentrations for vaccine formulations also include about 1, 5, 10, 15, 20, 25, 30, 35, 40, 45, 50, 55, 60, 65, 70, 75, 80, 85, 90, 95, and 100 μg/ml of the nanoparticles.

The skilled artisan will understand that the identity, number and size of the antigen component can vary greatly, depending on the disease or condition against which the subject is being vaccinated. However, suitable antigens include amino acids, peptides, polypeptides, proteins, nucleic acids, polynucleotides, bacteria and viruses, whether live, inactivated or dead, and cellular components of such bacteria and viruses including, for example, coat proteins.

The antigen and the nanoparticles may be conjugated together in the vaccine formulation or may be unconjugated in the vaccine formulation. Means for conjugating antigen and NPs are well known to the skilled artisan.

The vaccine formulations may also include a pharmaceutically acceptable carrier, diluent or excipient in the vaccine formulations, which will vary based on the identity of the antigens in the formulation, the means used to administer the formulation, the site of administration and the dosing schedule used. Suitable examples of carriers and diluents are well known to those skilled in the art and include water-for-injection, saline, buffered saline, dextrose, water, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes. The terms specifically exclude cell culture medium. Additional carriers include cornstarch, gelatin, lactose, sucrose, microcrystalline cellulose, kaolin, mannitol, dicalcium phosphate, sodium chloride, alginic acid, croscarmellose sodium, and sodium starch glycolate.

Excipients included in a formulation have different purposes depending, for example on the nature of the vaccine formulation and the mode of administration. Examples of generally used excipients include, without limitation: stabilizing agents, solubilizing agents and surfactants, buffers, antioxidants and preservatives, tonicity agents, bulking agents, lubricating agents, emulsifiers, suspending or viscosity agents, inert diluents, fillers, disintegrating agents, binding agents, wetting agents, lubricating agents, antibacterials, chelating agents, sweetners, perfuming agents, flavouring agents, coloring agents, administration aids, and combinations thereof.

The vaccine formulations of the present invention may also include an additional adjuvant. Suitable additional adjuvants include Freund's Complete and Incomplete Adjuvant, Titermax, Oil in Water adjuvants, as well as aluminum compounds where antigens, normally proteins, are physically precipitated with hydrated insoluble salts of aluminum hydroxide or aluminum phosphate. Other additional adjuvants include liposome-type adjuvants comprising spheres having phospholipid bilayers that form an aqueous compartment containing the antigen and protecting it from rapid degradation, and that provide a depot effect for sustained release. Surface active agents may also be used as additional adjuvants and include lipoteichoic acid of gram-positive organisms, lipid A, and TDM. Quil A and QS-21 (saponin-type adjuvants), monophosphoryl lipid A, and lipophilic MDP derivatives are suitable adjuvants that have hydrophilic and hydrophobic domains from which their surface-active properties arise. Compounds normally found in the body such as vitamin A and E, and lysolecithin may also be used as surface-active agents. Other classes of adjuvants include glycan analog, coenzyme Q, amphotericin B, dimethyldioctadecylammonium bromide (DDA), levamisole, and benzimidazole compounds. The immunostimulation provided by a surface active agent may also be accomplished by either developing a fusion protein with non-active portions of the cholera toxin, exotoxin A, or the heat labile toxin from E. coli. Immunomodulation through the use of anti-IL-17, anti IFN-γ, anti-IL-12, IL-2, IL-10, or IL-4 may also be used to promote a strong Th2 or antibody mediated response to the vaccine formulation.

The term “subject” is intended to mean an animal, such birds or mammals, including humans and animals of veterinary or agricultural importance, such as dogs, cats, horses, sheep, goats, and cattle.

A kit comprising the necessary components for immunomodulation, including a pharmaceutical formulation comprising one or both types of NPs and instructions for its use, is also within the purview of the present invention.

### EXAMPLES

### Reagents

Unless otherwise stated, the reagents used in the Examples disclosed herein were as follows. Bacterial lipopolysaccharide (LPS), phytohemagglutinin (PHA), and phorbol 12-myristate 13-acetate (PMA) were obtained from Sigma (St. Louis, Mo.). ROS levels were determined using 2-,7-dichlorodihydrofluorescein diacetate (DCF; Sigma). The NLRP3 inhibitor glybenclamide was purchased from Sigma.

### Synthesis of NPs

TiO₂ NPs were synthesized by a wet chemical synthesis, as described previously (Schanen et al. (2009) ACS Nano, 3, 2523-32). Briefly, a 50:50 mixture of ultrapure ethanol (Sigma) and deionized water (18.2 MΩ) was boiled to reflux. The pH of the boiling solution was adjusted to 3.0 with the addition of 1 N HCl. Titanium isopropoxide (Sigma) was added slowly to this refluxing mixture, which precipitated immediately to a white solution. The solution was then stirred at 85° C. for 4 h. The white solution was then cooled to room temperature and washed several times with ethanol until dry. The final preparation was mostly anatase (partially amorphous) TiO₂.

CeO₂ NPs were synthesized using a wet-chemical synthesis as described elsewhere (Karakoti et al. (2009) J. Am. Chem. Soc., 131, 14144-5). Briefly, cerium nitrate hexahydrate (99.999%, Sigma-Aldrich) was dissolved in deionized water (18.2 MΩ). A stoichiometric amount of hydrogen peroxide was added as an oxidizer, resulting immediately in the formation of cerium oxide NPs. NP powder was obtained by washing the precipitate of CeO₂ NPs several times with acetone and water to remove the surfactant used in the synthesis process. The solution was aged further to allow the slow reduction of surface cerium from the 4⁺ oxidation state to the 3⁺ oxidation state in acidic medium by maintaining the pH of the suspension below 3.5 with nitric acid.

### Nanoparticle Characterization

The physical characteristics of the TiO₂ and CeO₂ NPs were assessed because differences in NP preparation, dispersion, and agglomeration may affect their interaction with the immune system.

NPs were analyzed using high-resolution transmission electron microscopy (HRTEM; Philips 300 TECNAI, operated at 300 kV) to confirm the shape, size, and morphology of the NPs. HRTEM samples were prepared by dipping a polycarbon-coated copper grid in a dilute suspension of NPs dispersed in acetone. The surface area of the NPs was measured based on physical adsorption of ultra-high purity nitrogen gas at liquid nitrogen temperature on 100 mg of NPs using a Brunauer-Emmett-Teller (BET) Nova 4200e instrument (Quantachrome; Boynton Beach, Fla.). Samples were prepared in quartz tube and degassed at 240° C. in vacuo for 3 h before the measurement. The size of the NPs was determined by dynamic light scattering method using a Zetasizer Nano (Malvern Instruments, Worcestershire, UK). The physical characterization of the materials is reviewed in Table 2.

The sizes of the CeO₂ particles were ˜3 to ˜5 nm. In terms of chemistry, the particles had a Ce³⁺/Ce⁴⁺ ratio of −1.32. They had a positive surface charge of +17.78±0.5 mV.

TABLE 2
Physical properties of nanomaterials investigated

| | | Diam- | BET | Zeta | |
|---|---|---|---|---|---|
| | Preparation | eter | Surface | Potential | Crystal |
| Particles | Method | (nm) | (m²/g) | (mV)* | Structure |
| TiO₂ | HT-WCS^{a} | 7-10^{b} | 239 | −9.84 ± 0.19 | Anatase |
| CeO₂ | RT-WCS^{c} | 3-5^{b} | 90 | −10.01 ± 1.50 | Fluorite |

^{a}High temperature wet chemical synthesis.
^{b}Average diameter of NPs expressed as mean size ± SD nm.
^{c}Room temperature wet chemical synthesis.
*Zeta potential after 24 hrs in X-VIVO 15 culture media.

It was confirmed that both types of NP preparations were free of contaminating LPS (EU<0.05; data not shown). All NP preparations were confirmed negative for the presence of endotoxin contamination using the FDA-approved Endosafe LAL colorimetric and turbidimetric assay systems (Charles River Laboratories, Wilmington, Mass.).

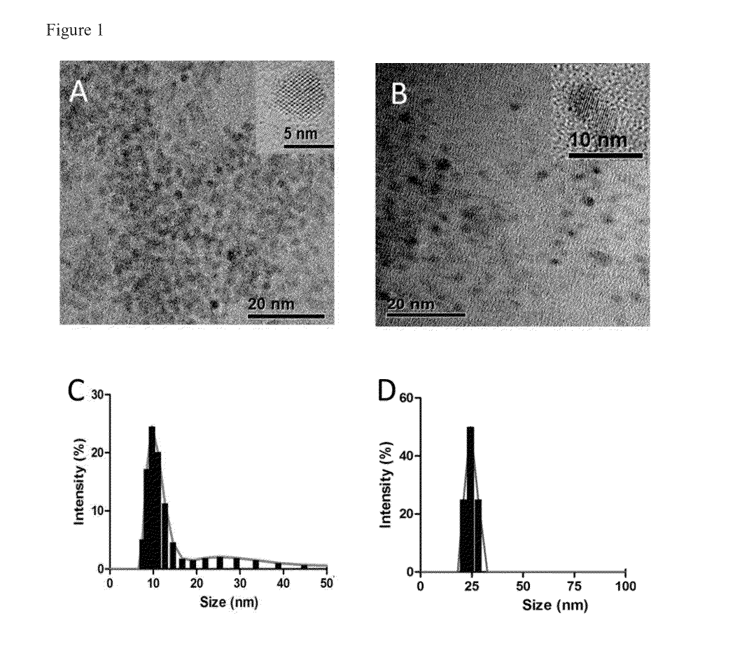

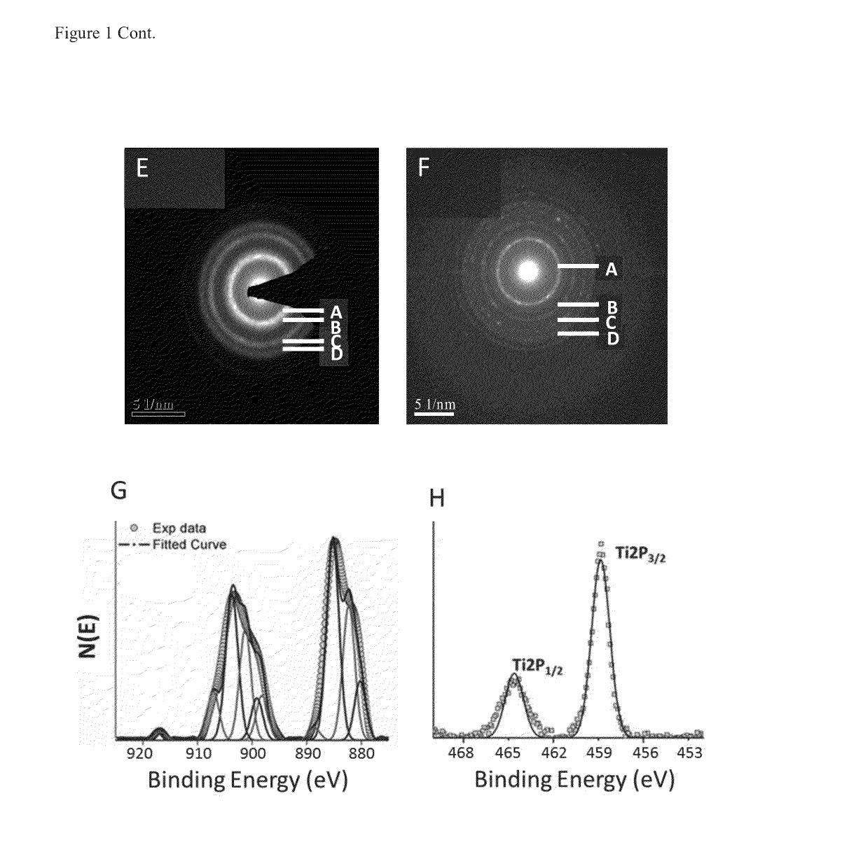

Size distribution was assessed by transmission electron microscopy (FIG. 1A, B) and dynamic light scattering (FIG. 1C, D), to examine NP agglomeration. The agglomeration tendency of the materials in culture medium was measured to demonstrate adequate material dispersion (FIG. 1). A low agglomeration tendency of both type NPs investigated was found after 24 h of incubating the NPs in X-VIVO 15 serum-free culture medium with size distributions of less than 25 nm in diameter on average (FIG. 1).

### Human Donors and PBMC Isolation

In relevant assays, PBMCs from healthy donors were used who provided informed consent. Blood collections were performed at Florida's Blood Centers (Orlando, Fla.) using standard techniques approved by their institutional review board. Within hours following their harvest from the donor, enriched leukocytes were centrifuged over a Ficoll-plaque PLUS (GE Healthcare, Piscataway, N.J.) density gradient (Moser et al. (2010) J. Immunol. Methods, 353, 8-19; Schanen & Drake (2008) J. Immunol. Methods, 335, 53-64). PBMCs at the interface were collected, washed, and cryopreserved in IMDM media (Lonza, Walkersville, Md.) containing autologous serum and DMSO (Sigma-Aldrich, St. Louis, Mo.).

### Generation of Cytokine-Derived DCs

DCs used in the relevant assays were prepared using a previously published methodology (Moser et al. (2010) J. Immunol. Methods, 353, 8-19). Briefly, monocytes were purified from total PBMCs by positive magnetic bead selection (Miltenyi Biotec, Cologne, Germany) and cultured for 7 days in X-VIVO 15 (Lonza) serum-free medium supplemented with GM-CSF (R&D Systems, Minneapolis, Minn.) and IL-4 (R&D Systems). In all assay conditions described below, treatments were delivered on day 6 followed by harvesting on day 7 for incorporation into the various assays.

### ROS Determination

DCs were cultured in 12-well dishes at a density of 2.5×10⁶ cells per well in 2.5 mL. The cultures were then treated with serial dilutions of TiO₂ NPs and CeO₂ NPs for 24 h. Subsequently, cultures were treated at room temperature for 30 min with DCF at a final concentration of 10 μM. Cells were washed of excess dye with DPBS, harvested using cell dissociation solution (Sigma), and washed again in DPBS. Fluorescence in the FITC channel from absorbed and oxidized DCF (indicating peroxide levels) was analyzed by flow cytometry using an LSR II (BD Pharmingen, San Diego, Calif.). The FlowJo software (Treestar, Ashland, Oreg.) was used for data analysis.

### DC Phenotyping

For flow cytometry analysis of surface molecule expression, DCs were washed in fluorescence-activated cell sorting buffer (FACS, 0.1% sodium azide and 0.1% bovine serum albumin in phosphate-buffered saline). Fc receptors were blocked with 10% mouse serum (Jackson ImmunoResearch, West Grove, Pa.) for 10 min at 4° C. to prevent nonspecific binding. DCs were then stained with a vital dye and incubated at 4° C. for 20 min. After washing away excess viability dye with PBS, the cells were then incubated with the appropriate antibody cocktail for 20 min on ice. The antibodies used in the staining panels include allophycocyanin-Cy7-labeled HLA-DR (LN3), eFluor 450-labeled CD14 (61D3), fluorescein isothiocyanate-labeled CD40 (5C3), phycoerythrin-labeled CD80 (2D10.4), allophycocyanin-labeled CD83 (HB15e), fluorescein isothiocyanate-labeled CD86 (IT2.2), peridinin chlorophyll protein (PerCP)-Cy5.5-labeled CD19 (SJ25C1), peridinin chlorophyll protein (PerCP)-Cy5.5-labeled CD3 (OKT3), allophycocyanin-labeled CD209 (LWC06), and phycoerythrin-Cy7-labeled CCR7 (3D12). CD14, CD11c, HLA-DR, CD40, CD19, CD3, CD80, CD83, CD86, CCR7 were purchased from eBioscience (San Diego, Calif.). CD209 was purchased from BD Pharmingen. Following staining, cells were washed in FACS buffer and immediately analyzed using a BD LSRII flow cytometer (Becton Dickinson), and data were analyzed using the FlowJo software (ver. 9.2; Tree Star).

### Cellular Uptake

Samples treated for 24 h with TiO₂ or CeO₂ NPs were harvested, washed, and placed in 70% nitric acid overnight to start the digestion process. Samples were then microwave-digested. The temperature was steadily increased to 200° C. over a 20-min period and held for 20 min at 200° C. Samples were then boiled down to less than 1 mL each and reconstituted in water to an exact volume of 10 mL. Titanium and cerium levels were assessed using inductively coupled plasma mass spectroscopy (ICP-MS).

### Luminex Cytokine Quantification Analysis

Supernatants from the treated DC culture wells and DC:T cell co-cultures were collected and analyzed for cytokine production using a Bioplex multiplexing array system (Bio-Rad, Hercules, Calif.) as described previously (Schanen et al. (2009) ACS Nano, 3, 2523-32).

### CD4⁺ T Cell Proliferation and Induction of T_{Regs}

Human CD4⁺ T cells were isolated from human peripheral mononuclear cells (PBMC) of healthy blood donors by positive selection using the EasySEP CD4⁺ T cell isolation kit II (Stem Cell Technologies, Vancouver, Canada). Purified CD4⁺ T cells were then carboxyfluorescein succinimidyl ester (CFSE)-labeled to follow proliferation and incubated either in the presence of the described NPs with or without PHA/PMA or without stimulation and left in culture for 5 days.

For T_{Reg} analysis, the T cells were co-cultured with DCs matured with TNFα and PGE₂ for 7 days as described previously (Banerjee et al. (2006) Blood, 108, 2655-61). The cells were harvested, and examined by flow cytometry using LIVE/DEAD AQUA (Pacific Orange; Invitrogen), CD4⁺ (Pacific Blue; eBioscience), CD25 (APC; eBioscience), Foxp3 (PE; BioLegend), and CFSE (FITC; Invitrogen) using a BD LSRII flow cytometer (Becton Dickinson), and data were analyzed using the FlowJo software (ver. 9.2; Tree Star).

Naïve CD4⁺ T Cell Allogeneic Stimulation Assay

DCs were either untouched, matured with a cocktail of TNF-α and PGE₂, as a positive control, or were exposed to various doses of NPs for 24 h prior to being harvested. The treated DCs were harvested and added at an optimized ratio of 1:400 to allogeneic naïve CD4⁺ T cells isolated using the EasySEP CD4⁺ T cell isolation kit II (Stem Cell Technologies) and labeled with CFSE (Invitrogen).

Here, PHA/PMA (1 μg/mL; 50 ng/mL) was used not only as a positive control for T cell proliferation, but was also added in combination with NPs additionally added to the co-culture wells where described. After 5 days, cultures were harvested and stained for CD25, CD3, and CD4 (eBioscience) and Live/Dead Aqua for viability (Invitrogen) and then assessed by flow cytometry using a BD Pharmingen LSR II, as described above. Supernatants were collected and analyzed for cytokines.

### Data Plotting and Statistical Analyses

Each experiment was repeated with at least three donors or more, as described in the figure legends. Analyzed statistical results were determined using a paired Student's t-test. Statistical significance was set at p<0.05. All graphs were produced using GraphPad Prism software (ver 5; La Jolla, Calif.).

### Example 1

### Nanoparticle Cytotoxicity

Over 4 million tons of pigmentary TiO₂ is consumed globally each year for uses including paints, papers, plastics, sunscreens, and cosmetics (Donaldson et al. (1996) Toxicol. Lett., 88, 293-8; Gilmour et al. (1997) Environ. Health Perspect., 105 (suppl 5), 1313-7; Goncalves et al. (2010) Toxicol. In Vitro, 24, 1002-8; Jin et al. (2008) Chem. Res. Toxicol., 21, 1871-7; Sayes et al. (2006) Toxicol. Sci., 92, 174-85; Vamanu et al. (2008) Int. J. Nanomedicine, 3, 69-74). CeO₂ NPs also have wide applications, from solar cells, fuel cells, gas sensors, oxygen pumps, and refining glass/ceramic production to proposed biomedical applications (Celardo et al. (2011) Nanoscale, 3, 1411-20; Celardo et al. (2011) J. Exp. Ther. Oncol., 9, 47-51).

Beyond their broad commercial appeal and thus heightened risk for exposure, these two materials were chosen because they possess opposite redox behavior: CeO₂ NPs (oxidant-scavenging) and TiO₂ NPs (oxidant-generating), providing an opportunity to investigate in parallel the impact of opposite catalytic NPs towards immunity.

While previous studies have largely focused on the inflammatory effect of NPs using cell lines or phagocytic cells (Valles et al. (2006) Biomaterials, 27, 5199-211), the effects of NPs on DCs were investigated because they are pivotal in both innate and adaptive immunity, being solely capable of activating naïve CD4⁺ helper T cells, a key support cell for inducing humoral and cellular immunity and long-lived memory.

Before assessing immunological responses resulting from the interaction of NPs with DCs, it was first determined whether CeO₂ and TiO₂ NPs had cytotoxic effects on human DCs in the range of concentrations used.

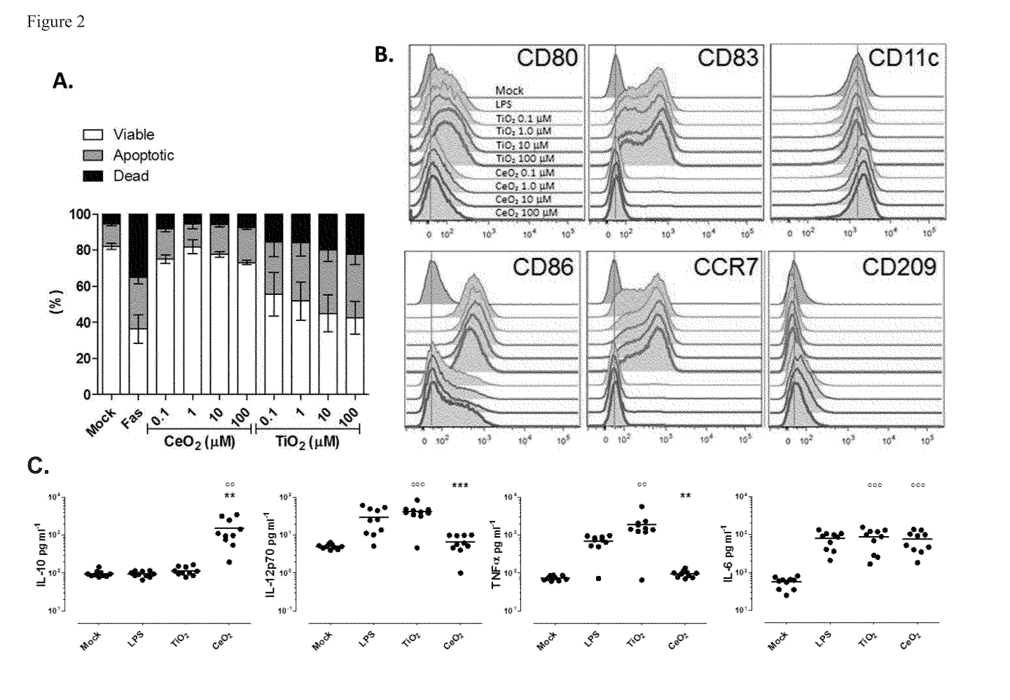

Following short-term treatment of the DCs with NPs, the cells were assessed with the fluorescent apoptotic dye (Po-Pro) in combination with a vital dye (7-AAD) to discriminate between live, dead, and apoptotic cells. No increased cell death or apoptosis was observed in DCs exposed to CeO₂ NPs for 24 h, while DCs treated with TiO₂ NPs for the same time period had an appreciable increase in the number of apoptotic and dead cells, in a dose-dependent manner (FIG. 2A). Higher doses (100 μM) of TiO₂ induced increased cell death and apoptosis, compared with CeO₂ NP-treated DCs (FIG. 2). While the findings on TiO₂ NPs cytotoxicity in human DCs are consistent with previous work and the observations of others using cell lines (Schanen et al. (2009) ACS Nano, 3, 2523-32; Xu et al. (1998) Supramolecular Science, 5, 449-451; Zhang & Sun (2004) World J. Gastroenterol., 10, 3191-3; Bar-Ilan et al. (2011) Nanotoxicology, 0, 1-10), it is reported here for the first time that CeO₂ NPs were not cytotoxic to human DCs.

### Example 2

### Phenotypic Maturation of DCs

While metal oxide NPs may lack multiple danger signals common to complex biological immunogens, distinct redox-surface chemistry can provide the mechanisms necessary for innate activation via modulation of messengers like reactive oxygen species (ROS), for which innate danger sensors, such as the NLRP3 inflammasome, exist (Yazdi et al. (2010) Proc. Natl. Acad. Sci. USA 107, 19449-54). As part of the innate response, DCs undergo a maturation process, which may be broadly qualified by two classic physiological features: increased expression of surface costimulatory and MHC molecules (CD80, CD86, HLA-DR) and changes in chemokine receptor expression (CCR7).

DCs treated with as little as 1 μM TiO₂ NPs induced expression of CD80 and CD86 and increased the expression of HLA-DR, comparable to the level induced by the positive control, LPS. On the other hand, FIG. 2B shows upregulation of CD83, a phenotypic hallmark of DC maturation, only at a higher TiO₂ dose (100 μM).

Interestingly, 24-h exposure of the DCs to CeO₂ NPs had no effect on CD83, CD80, CD86, or HLA-DR expression. In concert with the observed costimulatory marker upregulation, it was demonstrated that exposure to TiO₂ NPs, but not CeO₂ NPs, induced increased expression of CCR7 (FIG. 2B). CCR7 expression is significant because of its role in DC migration towards lymph nodes where antigen presentation occurs; thus, materials that upregulate CCR7 expression may enhance immunity.

### Example 3

### Induction of DC Cytokine Expression

In addition to the upregulation of costimulatory markers, DCs may also secrete anti- or pro-inflammatory cytokines, which can direct the immune response. Thus, the impact TiO₂ and CeO₂ NPs on DC cytokine secretion was studied. DCs were incubated in the presence or absence of a range of CeO₂ or TiO₂ NPs for 24 h followed by quantification of key cytokine levels in culture supernatants (FIG. 2C).

DCs treated with TiO₂ NPs increased their production of IL-12, p70 and TNF-α, while CeO₂ NPs enhanced IL-10 cytokine secretion (FIG. 2C). The proinflammatory cytokine production observed following TiO₂ NP exposure was consistent with the mature phenotype demonstrated in FIG. 2B. The opposite surface chemistry, low porosity, and decreased surface area are among the physicochemical features that differ between the CeO₂ and TiO₂ NPs and that may contribute to the impact profile of CeO₂ NPs on innate immunity. Similar to this finding (FIG. 2B) are studies where antioxidants (reductive molecules) were shown to increase IL-10 production (Chauveau et al. (2005) Blood, 106, 1694-702; Wang et al. (1995) J. Biol. Chem., 270, 9558-63), which can be an immunosuppressant (Chen et al. (2007) J. Immunol., 179, 6009-15).

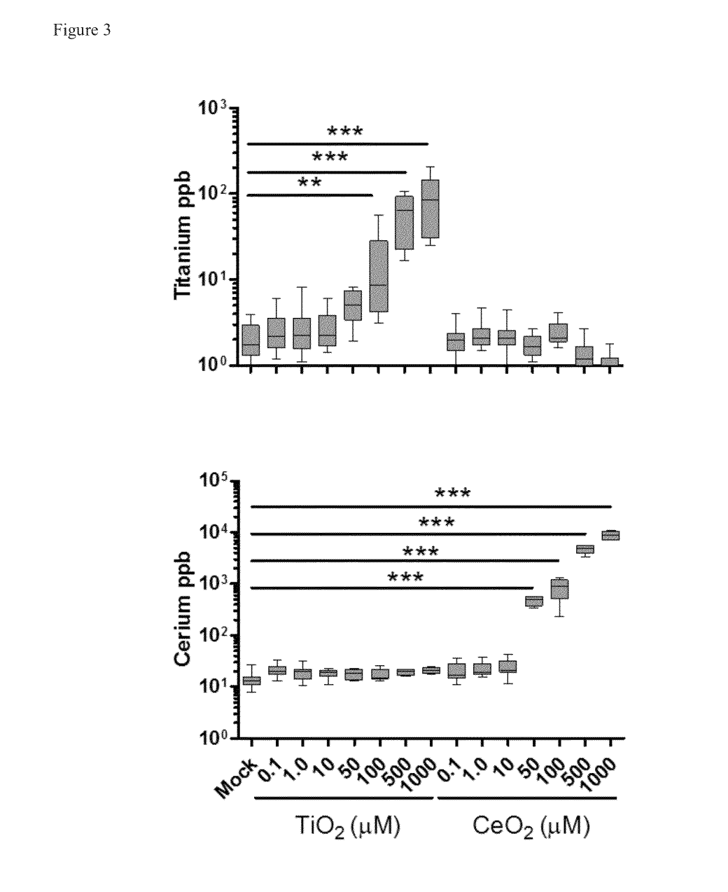

Whether the difference in effect between the two types of NPs was attributable to differences in NP uptake was examined next. ICP-MS were used to examine the presence of metals within the treated DCs because this methodology has the sensitivity to detect NPs within the single-digit part-per-billion range (FIG. 3). It was determined that uptake was dose-dependent and detectable by ICP-MS at 50 μM for both NPs, leading to the conclusion that the difference between the NP surface chemistries had no influence on the frequency of uptake.

In previous studies, uptake of TiO₂ was demonstrated only with much higher dosing (5 mg/mL, 62.5 mM) reaching only as low as 250 μM in another study (Churg et al. (1998) Am. J. Physiol., 274, L81-6; Teste et al. (2011) Lab Chip, 11, 4207-13). Similarly, CeO₂ uptake has only been demonstrated with higher dosing than that used in the present work (Zhu et al. (2011) Biosens. Bioelectron., 26, 4393-8; Di Gioacchino et al. (2011) Int. J. Immunopathol. Pharmacol. 24, 65S-71S).

### Example 4

### Intracellular Assessment of ROS

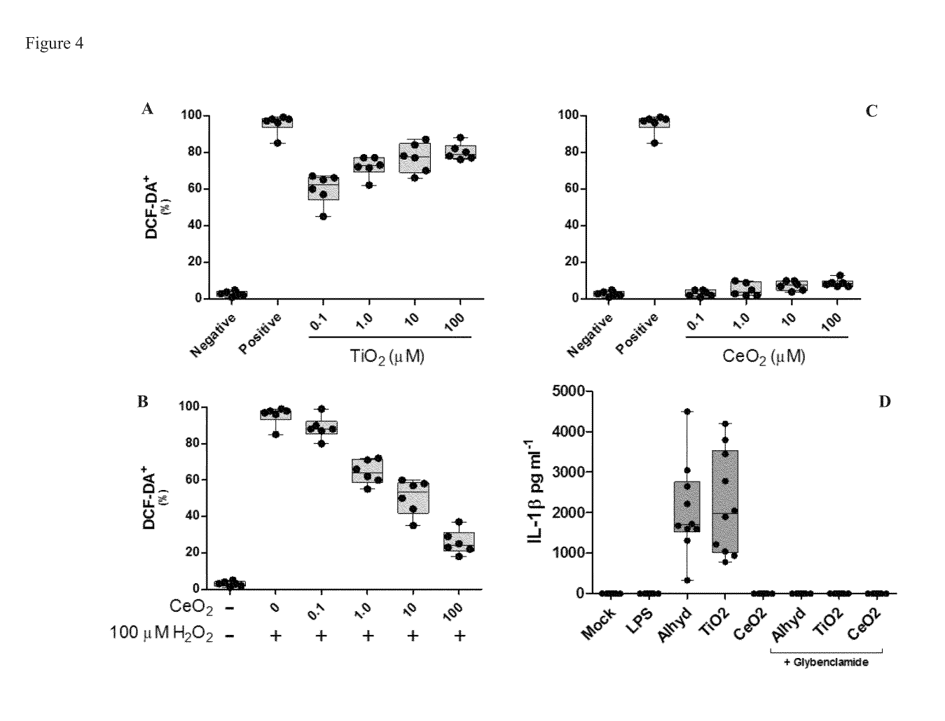

Because it was suspected that the two catalytic NPs could differentially trigger reactive oxygen species (ROS), ROS levels in DC cultures were investigated following NP treatment. Intracellular oxidative stress was analyzed using the DCF-DA dye, which fluoresces on contact with ROS. FIG. 4 shows that TiO₂ NPs induced human DCs to generate higher levels of ROS, comparable to H₂O₂, a positive control used to induce detectable ROS levels in culture.

In contrast, CeO₂ NP-treated DCs showed little or no production of ROS (FIG. 4). Furthermore, these findings are consistent with work suggesting that CeO₂ NPs have antioxidant properties capable of mitigating ROS production, as demonstrated by reduced ROS levels observed in DCs pre-treated with CeO₂ and then incubated with H₂O₂ (FIG. 4B).

While ROS acts through a variety of downstream pathways to regulate/potentiate immune reactions, perhaps its most important feature is the ability to activate the NLRP3 inflammasome. The NLRP3 inflammasome is a notable NLR family member that works to rapidly mobilize immune responses to cognate danger signals.

Because IL-1β has been used routinely as a readout of inflammasome activation (Yazdi et al. (2010) Proc. Natl. Acad. Sci. USA 107, 19449-54), whether TiO₂ NPs could activate the NLRP3 inflammasome in human DCs was examined via IL-1β detection (FIG. 4C). Indeed, it was found that TiO₂ NP-treated DCs did secrete IL-1β, consistent with prior studies demonstrating that TiO₂ NPs activate the NLRP3 inflammasome in mice (Yazdi et al. (2010) Proc. Natl. Acad. Sci. USA 107, 19449-54). A selective NLRP3 inhibitor, glybenclamide (50 μM) (Lamkanfi et al. (2009) J. Biol. Chem. 284, 20574-81), was used in tandem with TiO₂ NPs to determine whether TiO₂ NPs could act through other NLR inflammasome members resulting in IL-1β secretion. It was found that when TiO₂ NPs and glybenclamide were co-administered, IL-1β production was abolished (FIG. 4D). In contrast to the activation of NLRP3 by TiO₂NPs, it was observed that CeO₂ NPs were unable to instigate IL-1β production.

On the basis that TiO₂NPs were capable of upregulating CD80, CD83, CD86, HLA-DR, and CCR7 in combination with increased ROS generation, concomitant with NLRP3 activation, TiO₂ NPs are thought to potentiate human DC activation, while CeO₂ NPs do not engage DCs to upregulate surface receptors, but do have a stimulatory effect on DCs, to generate IL-10 and potentially mitigate oxidative stress.

### Example 5

### NPs Drive CD4⁺ T Cell Proliferation and T_{H}1/T_{H}2 Polarization

Whether TiO₂ and CeO₂ NPs have an effect on adaptive cellular immunity was examined. To test this, whether NPs could modulate T cell activation or lymphoproliferation was studied. Isolated CD4⁺ T cells labeled with CFSE (proliferation detection dye) were used to monitor proliferative responses to the NPs. On their own, TiO₂ NPs had a modest effect on lymphoproliferation.

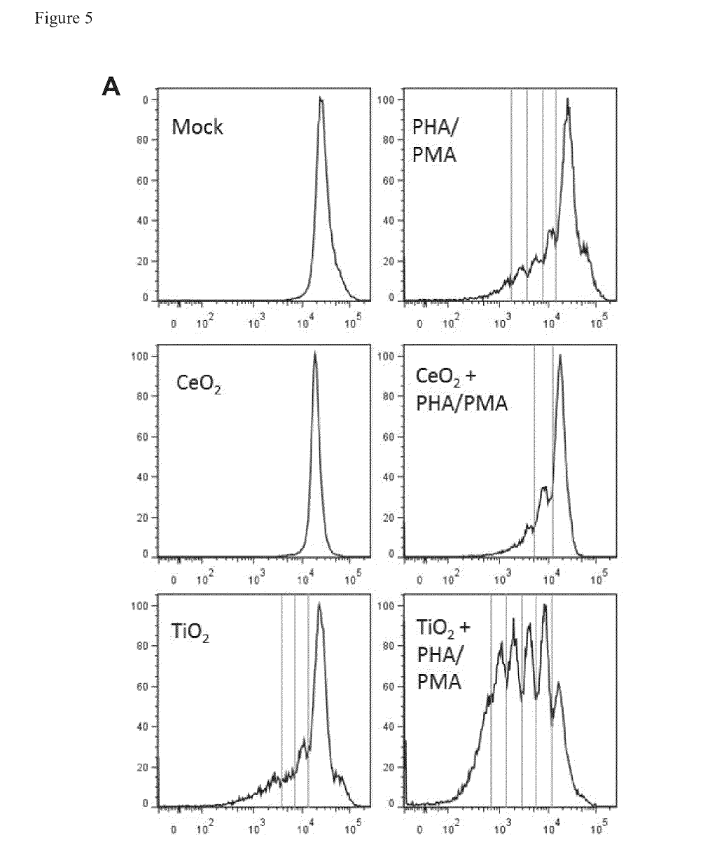

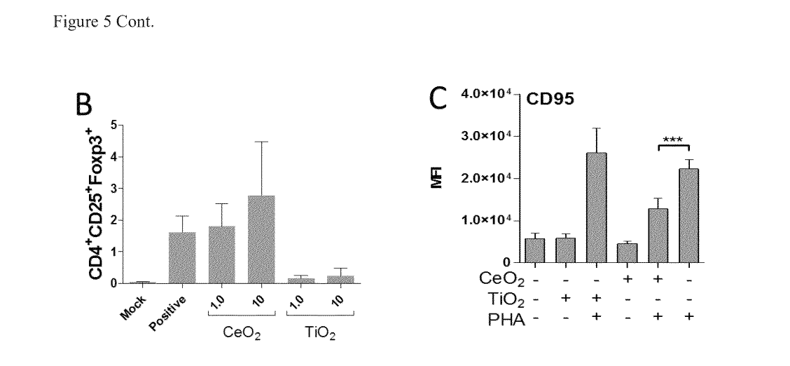

The effects of NPs on T cells when co-administered with strong mitogens/nonspecific stimulators of T cells (PHA/PMA) were examined. When NPs were co-administered with PHA/PMA, TiO₂ NPs amplified the proliferative response while CeO₂ NPs moderately reduced the proliferative response (FIG. 5A).

Considering the innate responses induced by CeO₂ NPs, the influence CeO₂ NPs on the induction of regulatory T cells (T_{Reg}) was next investigated, as determined by staining for Foxp3, a specific marker of T_{Regs} (FIG. 5B). While the capacity for NPs to induce T_{Regs} has recently been shown (Park et al. (2011) Mol. Pharm., 8, 143-52), this was observed using PLGA NPs decorated with leukemia inhibitory factor.

Here, the capacity of naked CeO₂ NPs to induce T_{Regs} differentiation was demonstrated. Additionally, because the expression of CD95 in resting T cells has been shown to increase under stress or in disease conditions, modulation of this receptor, which has implications in co-stimulatory and effector function (Paulsen et al. (2011) 18, 619-31), was examined. FIG. 5C shows that there is a strong correlation for reduced CD95 expression in CeO₂ NP-treated Th cells, compared with the mitogen control or TiO₂ NP treatment.

To further understand the impact these NPs have towards adaptive immunity, the influence of both NPs on effector T cell responses in an allogeneic stimulation assay was investigated. Here, DCs were left untouched (iDC), matured with cytokine cocktail (mDC), or primed with either NP and transferred to allogeneic purified T cell cultures. The matured DCs were added as an activated DC control for comparison. Two additional groups were prepared for the above conditions where the co-culture was incubated with additional NP or NP+PHA to identify whether the persistence of NPs or NP+PHA in the co-culture had a cumulative effect.

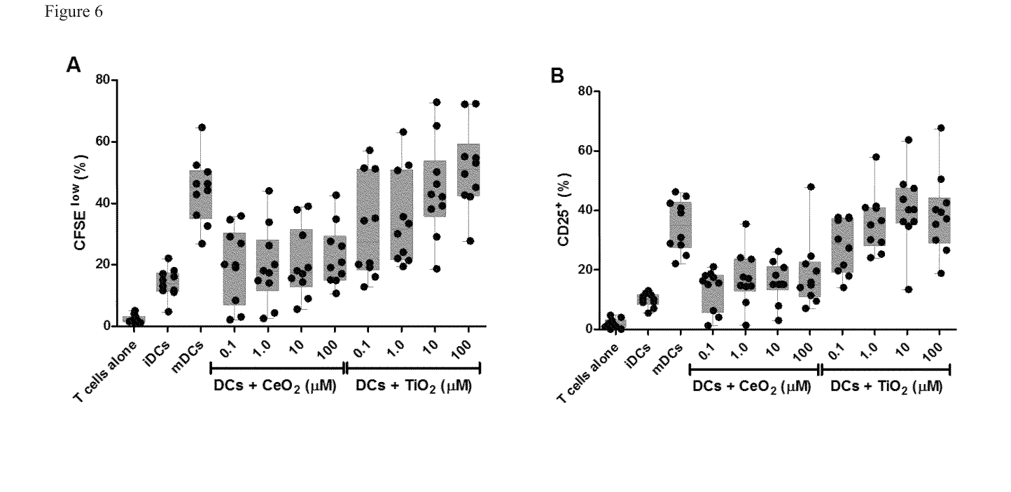

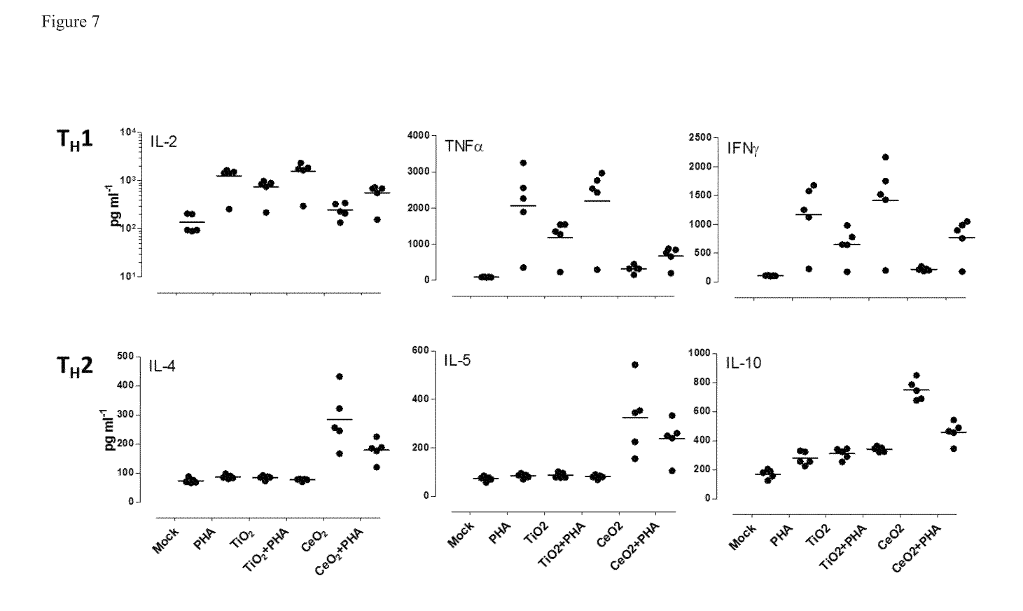

Although the CeO₂ NP-treated DCs had little influence on allogeneic naive CD4+ T cell proliferation, TiO₂ NP-treated DCs boosted the magnitude of the proliferative response (FIG. 6). As well, it was observed that both particles triggered cytokine responses, but the profiles were nearly opposite: TiO₂ NPs-pulsed DCs triggered a pro-inflammatory T_{H}1-biased cytokine response (IL-2, IFN-γ) while DCs pulsed with CeO₂ NPs induced a naive T cell response dominated by T_{H}2 cytokines (IL-4, IL-5, and IL-10) that are predominately anti-inflammatory and promote humoral-skewed responses. Beyond their capacity to participate in the induction of a T_{H}2-biased T cell response, the CeO₂ NPs were even capable of eliciting the production of IL-4, IL-5 and IL-10 in T cell co-cultures stimulated with a strongly T_{H}1-biasing mitogen (FIG. 7). While it might have been anticipated that a well-described pro-inflammatory particle like TiO₂ could drive a type 1 immune response, the response profile induced by CeO₂ NPs, including IL-10 secretion by DCs (FIG. 2) and T_{H}2 polarization (FIGS. 6 and 7), suggest a unique functional property of metallic antioxidant NPs that has not previously been described.

While NP-induced T_{H} polarization has been observed by other groups (Conway et al. (2001) Vaccine, 19, 1940-50; He et al. (2000) Clin. Diagn. Lab. Immunol., 7, 899-903; Liu et al. (2009) Biomaterials, 30, 3934-45; Lutsiak et al. (2006) J. Pharm. Pharmacol., 58, 739-47), these results differed greatly from previous observations in that catalytic NPs were investigated using primary human T cells, revealing differential effects between redox materials, specifically that CeO₂ NPs drive strongly towards a T_{H}2 polarization program.

Cumulatively, the observed absence of toxicity in the effective dose range, IL-10 secretion by DCs (FIG. 2C), upregulation of T_{Regs}, modulation of T cell proliferation, and T_{H}2 polarization (FIG. 5-7) suggest unique functional properties of CeO₂ NPs that may useful across a wide range of NP therapeutic applications.

All documents, publication, manuals, article, patents, summaries, references and other materials cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

### Example 6

### Adjuvant Effect of TiO₂ and CeO₂ Nanoparticles

The effectiveness of the two types of nanoparticles as adjuvants, for example in vaccine formulations, was tested.

Several formulations were tested, as shown in Table 3 and included: (1) CMV-gB and CeO₂ NPs; (2) CMV-gB and TiO₂ NPs; (3) CMV-gB and MF59.

TABLE 3

| Test For- | |
|---|---|
| mulations | Components |
| A | CMV-gB (2 μg) |
| B | CMV-gB (2 μg) + MF59 [2% squalene] |
| C | CMV-gB (2 μg) + TiO₂ NPs (5 μg) |
| D | CMV-gB (2 μg) + TiO₂ NPs (0.05 μg) |
| E | CMV-gB (2 μg) + CeO₂ NPs (10.75 μg) |
| F | CMV-gB (2 μg) + CeO₂ NPs (0.1075 μg) |
| G | CMV-gB (2 μg) + TiO₂ NPs (5 μg) + CeO2 NPs (10.75 μg) |
| H | CMV-gB (2 μg) + TiO₂ NPs (0.05 μg) + CeO2 NPs |
| | (10.75 μg) |
| I | PBS |

The test formulations were administered to C57Bl6 mice, 10 mice per groups, IM 50 μl on Day 0 and Day 21. Blood and splenocyte sampling was conducted on Day 35. IgG1/IgG2c antibodies were assayed in serum by ELISA. IFNγ and IL5 were assayed on CMV-gB-restimulated splenocyte supernantents by CBA.

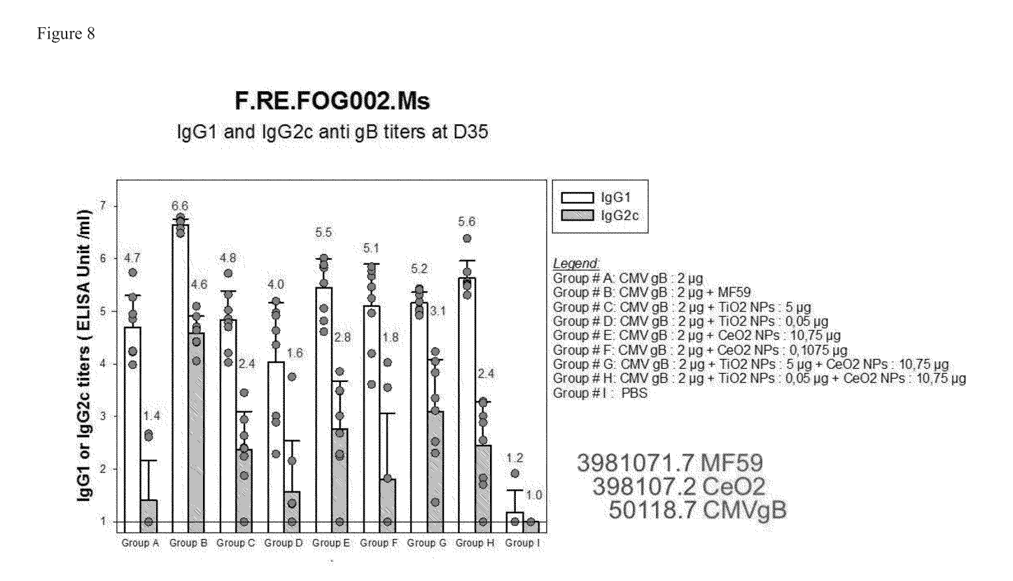

The results showed that IgG1 and IgG2c levels were increased 8 fold in mice injected with the test formulation comprising CMV-gb/CeO₂ while the levels increased 80 fold in mice injected with CMV-gb/MF59 (FIG. 8).

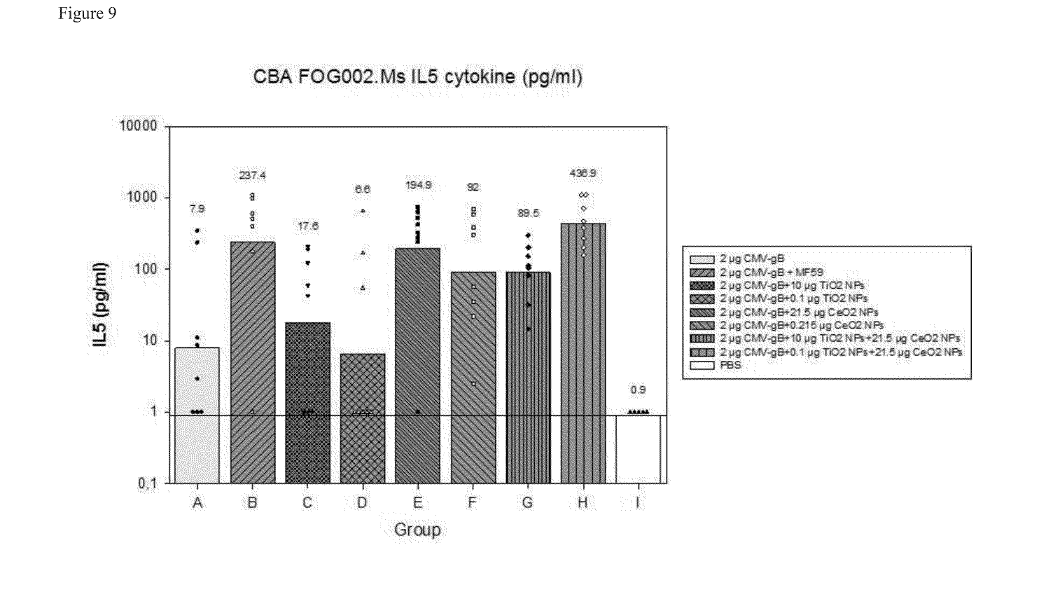

The results further showed that IL-5 levels increased 24.7 fold in mice injected with CMV-gb/CeO₂ while the levels increased 30 fold in mice injected CMV-gb/MF59 (FIG. 9).

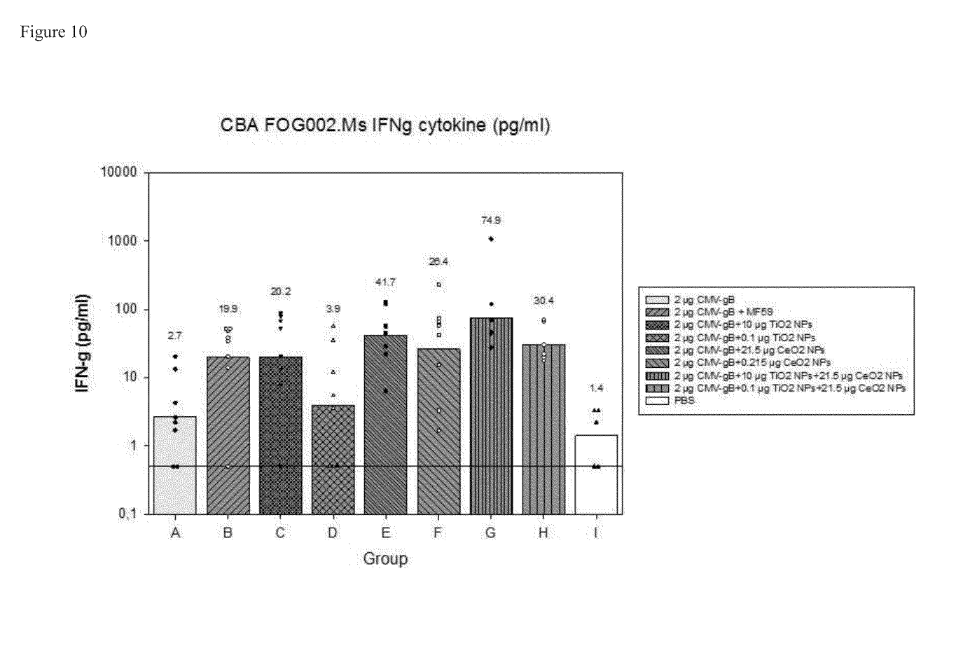

The results also showed that IFNγ levels increased 15.3 fold in mice injected with CMV-gb/CeO₂ while the levels increased 7.3 fold in mice injected CMV-gb/MF59 (FIG. 10).

### Example 7

### Adjuvant Effect of TiO₂ Nanoparticles

A further test of the effectiveness of TiO₂ nanoparticles as an adjuvant was tested. Several formulations were tested, as shown in Table 4.

TABLE 4

| Test For- | |
|---|---|
| mulations | Components |
| A | HIV p24 (5 μg) |
| B | HIV p24 (5 μg) + AlOOH (0.6 mg) [standard Alum |
| | hydroxide] |
| C | HIV p24 (5 μg) + AlPO4 (0.6 mg) [standard Alum |
| | phosphate] |
| D | HIV p24 (5 μg) + Mg/Al hydrotalcite (0.6 mg) |
| E | HIV p24 (5 μg) + Mg/Fe hydrotalcite (0.6 mg) |
| F | HIV p24 (5 μg) + CaPO4 (0.6 mg) |
| G | HIV p24 (5 μg) + Mg/Al decarbonated hydrotalcite (0.6 mg) |
| H | CMV-gB (2 μg) + TIO2 NPs |

The test formulations were administered to BALB/c mice, 6 mice per groups, SC 200 μl on Day 0 and Day 21. Bleeding on Day 35 and splenocyte sampling was conducted on Day 37. IgG1/IgG2a antibodies were assayed in serum by ELISA. IFNγ and IL5 were assayed on HIV p24-restimulated splenocyte supernantents by ELISA.

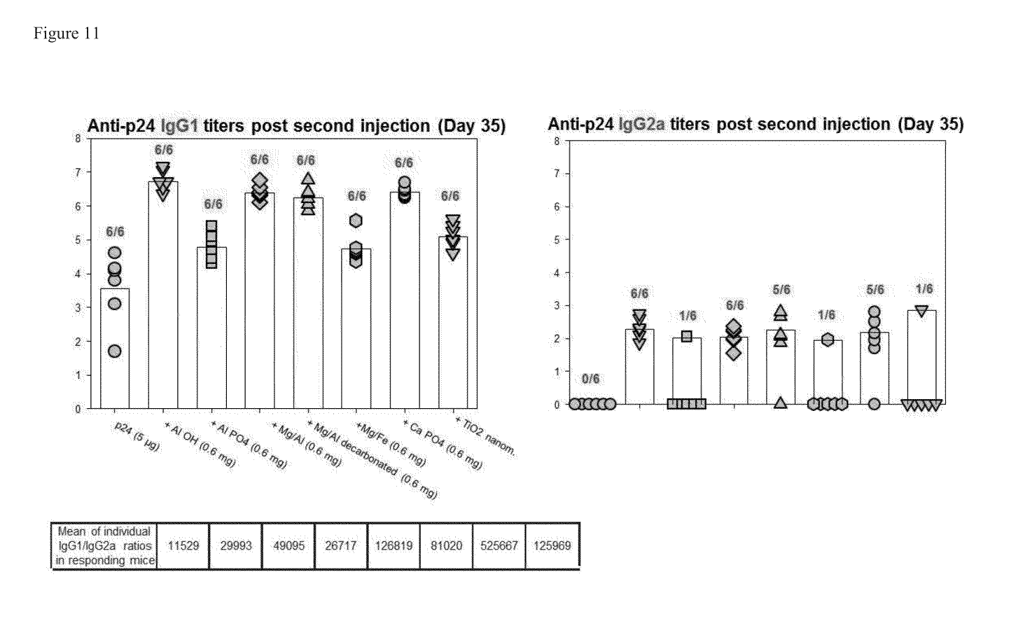

The results showed TiO₂ had the capacity to induce IgG1 and IgG2a antibody responses similar to Alum adjuvant formulations when delivered with HIV-p24 as an antigen (FIG. 11).

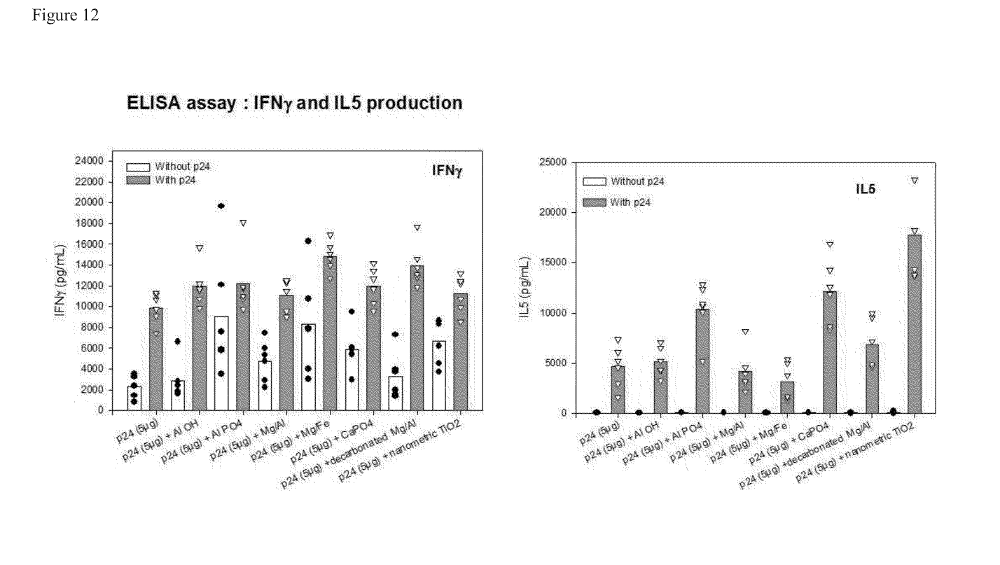

The results further showed that TiO₂ had the capacity to induce similar IFN-gamma responses as compared to standard Alum formulations and greater immunogenicity in context of IL-5 production when delivered with HIV-p24 as an antigen (FIG. 12).

While the invention has been described with reference to certain particular embodiments thereof, those skilled in the art will appreciate that various modifications may be made without departing from the spirit and scope of the invention. The scope of the appended claims is not to be limited to the specific embodiments described.

1. A method of modulating an immune response in a subject comprising administering a pharmaceutical formulation comprising nanoparticles of CeO₂ to a subject in need thereof in an amount sufficient to modulate an immune response in the subject.

2. The method of claim 1, wherein production of antigen presenting cells is modulated.

3. The method of claim 2, wherein the antigen presenting cells are dendritic cells.

4. The method of claim 1, wherein production of CD4⁺ T helper cells is modulated.

5. The method of claim 1, wherein production of CD8⁺ T helper cells is modulated.

6. The method of claim 1, wherein T_{H}2-type immune response is modulated and wherein the modulation may be stimulation or suppression.

7. A method of inducing a T_{H}2 T cell response in a subject comprising administering a pharmaceutical formulation comprising nanoparticles of CeO₂ to a subject in an amount sufficient to induce a T_{H}2 T cell response in the subject.

8. The method of claim 7, wherein the T_{H}2 T cell response is a T_{H}2-polarized T cell response.

9. The method of claim 7, wherein the T_{H}2 T cell response is production of T_{H}2 T cell cytokines.

10. A method of inducing dendritic cell (DC) cytokine production in a subject comprising administering a pharmaceutical formulation comprising nanoparticles of CeO₂ to a subject in an amount sufficient to induce DC cytokine production in the subject.

11. The method of claim 10, wherein DCs are induced to produce one or more of the following cytokines: IL-10, IL-1beta, IL-6, IL-7, IL-12 (p70), IL-15, IL-18, TNF-alpha, TGF-beta.

12-14. (canceled)

15. The method of claim 1, wherein the pharmaceutical formulation comprising nanoparticles of CeO₂ comprises between about 10 μg/ml and 100 μg/ml nanoparticles.

16. The method of claim 1, wherein the pharmaceutical formulation comprising nanoparticles of CeO₂ is a volume of between about 0.25 ml and 1.0 ml.

17. The method of claim 1, wherein the pharmaceutical formulation is administered to the subject via an intraperitoneal, intravenous, oral, or subcutaneous route.

18-23. (canceled)

24. The method of claim 7, wherein the pharmaceutical formulation comprising nanoparticles of CeO₂ comprises between about 10 μg/ml and 100 μg/ml nanoparticles.

25. The method of claim 7, wherein the pharmaceutical formulation comprising nanoparticles of CeO₂ is a volume of between about 0.25 ml and 1.0 ml.

26. The method of claim 7, wherein the pharmaceutical formulation is administered to the subject via an intraperitoneal, intravenous, oral, or subcutaneous route.

27. The method of claim 10, wherein the pharmaceutical formulation comprising nanoparticles of CeO₂ comprises between about 10 μg/ml and 100 μg/ml nanoparticles.

28. The method of claim 10, wherein the pharmaceutical formulation comprising nanoparticles of CeO₂ is a volume of between about 0.25 ml and 1.0 ml.

29. The method of claim 10, wherein the pharmaceutical formulation is administered to the subject via an intraperitoneal, intravenous, oral, or subcutaneous route.
